(19) United States
(12) Patent Application Publication
(10) Pub. No.: US 2009/0325291 A1
(43) Pub. Date: Dec. 31, 2009

(54) METHOD OF PREPARING siRNAs FOR SELECTIVE INHIBITION OF TARGET mRNA ISOTYPES

(75) Inventors: Seong Min Park, Daejeon (KR); Young Joo Kim, Daejeon (KR); Young Chul Choi, Daejeon (KR); Han Oh Park, Daejeon (KR); So Rim Choung, Daejeon (KR)

Correspondence Address: ROTHWELL, FIGG, ERNST & MANBECK, P.C., 1425 K STREET, N.W., SUITE 800, WASHINGTON, DC, 20005, US

(73) Assignee: BIONEER CORPORATION, Daejeon (KR)

(21) Appl. No.: 12/304,707

(22) PCT Filed: Jun. 12, 2007
(86) PCT No.: PCT/KR2007/002827

§ 371 Date: Dec. 12, 2008

(30) Foreign Application Priority Data
Jun. 13, 2006 (KR) 10-2006-0052904

(51) Int. Cl. C12N 5/02; G01N 33/48; C07H 21/02

(52) U.S. Cl. 435/375; 702/19; 536/24.5

(57) ABSTRACT

A method of preparing siRNAs for selective inhibition of target mRNA isotypes comprises: dividing target mRNA isotypes intended to inhibit the expression thereof and non-target mRNA isotypes from the mRNA isotypes of a gene; allotting a common location information region (A) of exons on genome DNA corresponding to the target mRNA isotypes; allotting a location information region (B) present specifically in exons of genome DNA corresponding to target mRNAs by excluding the location information region of exons on genome DNA corresponding to non-target mRNA from the location information region (A); determining base sequences in the target mRNAs corresponding to the location information region (B); and obtaining siRNA sequences for inhibiting the determined base sequences specifically. The method of the present invention can be used to prepare siRNAs for selective inhibition of specific target mRNA isotypes in a gene having several isotypes by alternative splicing, and enables siRNA design for all the genes in genome, making good tool for functional genomics study.

### FIELD OF THE INVENTION

The present invention generally relates to a method of preparing siRNAs, and more specifically, to a method of designing siRNAs for inhibiting selectively the expression of specific target mRNA isotypes in a gene having several isotypes and specifically inhibiting the expression of target mRNAs using the siRNAs.

### DESCRIPTION OF THE RELATED ART

RNA interference (or RNAi) refers to inhibition of protein synthesis by degradation of target mRNAs in cytoplasm using a specific double-stranded RNA (dsRNA) having the same base sequence with the target mRNAs. After the RNAi is first found in C. elegans (nematode) in 1998 by Fire and Mello, it has been reported that RNAi phenomenon occurs in fruitfly (Drosophila), Trypanosoma (kind of flagellate), vertebrate and so on (Tabara H, Grishok A, Mello C C, Science, 282 (5388), 430-1, 1998). When the dsRNA is introduced into human cells, an antiviral interferon pathway is induced so that it is difficult to find an RNAi effect in human cells. However, it has been reported in 2001 by Elbashir and Tuschl that when a small dsRNA of 21 nucleotide (nt) length is introduced into human cells, the antiviral interferon pathway does not occur and the introduced dsRNA decomposes target mRNAs specifically (Elbashir, S. M., Harborth, J., Lendeckel, W., Yalcin, A., Weber, K., Tuschl, T., Nature, 411, 494-498, 2001; Elbashir, S. M., Lendeckel, W., Tuschl, T., Genes & Dev., 15, 188-200, 2001; Elbashir, S. M., Martinez, J., Patkaniowska, A., Lendeckel, W., Tuschl, T., EMBO J., 20, 6877-6888, 2001). Thereafter, the dsRNA of 21 nt length has been spotlighted as a new tool for functional genomics, and it has been called as small interfering RNA (siRNA). The small interfering RNA (siRNA and microRNA) was listed as No. 1 of the Breakthrough of the Year in the Science journal (Jennifer Couzin, BREAKTHROUGH OF THE YEAR: Small RNAs Make Big Splash, Jennifer Couzin, Science 20 Dec. 2002: 2296-2297).

The RNAi has several advantages as a tool for therapeutics and functional genomics in comparison of the conventional antisense RNAs. First, the conventional antisense RNA requires a lot of time and cost to perform experiments following synthesizing many antisense RNAs in order to obtain efficient target base sequences while the siRNA requires relatively small number of experiments to obtain efficient siRNAs because the efficiency of siRNA can be predicted through several algorithms. Second, it is reported that the siRNA (RNAi) can inhibit gene expression effectively in lower concentrations than the conventional antisense RNA. It means that when the siRNA is used for research, smaller amounts can be used, and when the siRNA is used for medical treatment, the siRNA can be very effective. Third, the inhibition of gene expression by RNAi is a mechanism that occurs naturally in a living organism, and its process is undergoing very specific manner.

An RNAi experiment includes highly efficient siRNA design (target site selection), cell culture assay (quantification of target mRNA reduction, selection of the most efficient siRNA), animal experiment (stability, modification, delivery, pharmacokinetics, toxicology) and clinical test. Of these steps, selection of highly efficient target base sequences and delivery of the siRNA into target tissues (drug delivery) are the most important. The highly efficient target base sequence is required because each base sequence has different siRNA efficiency, and highly efficient siRNA shows clear experimental results and can be used as a therapeutic agent. Searching a target base sequence includes a computer-based calculation method and an experimental method. The experimental method includes the steps of making a target mRNA by in vitro transcription and finding a base sequence that can be hybridized with the mRNA well. However, the structure of mRNA made in in vitro transcription may be different from that in cells. Also, since several proteins can be combined to the mRNA in the cells, a result obtained by the experiment using in vitro transcript may not reflect an actual result. Therefore, it is important to develop an algorithm of searching an efficient siRNA and this can be deduced by considering several variables that remove inefficient siRNA sequences.

Meanwhile, it has been well known that an alternative splicing in a cell where the siRNA acts serves as an important role in variety of proteins expressed from a genome (Graveley, B. R., Trends Genet., 100-107, 2001). For example, it has been reported that 74% of human genes has a alternatively spliced mRNA (Johnson, J. M., et al., Science (302), 2141-2144, 2003), and that Drosophila also has various alternatively spliced mRNAs (Schmucker, D., et al., Cell(101), 671-684, 2000). As a statistical result of mRNAs starting with NM_ in Refseq items of databases of the National Center for Biotechnology Information (NCBI) homepage (http://www.ncbi.nlm.nih.gov) and the genome bioinformatics homepage of University of California Santa Cruz (UCSC) (http://www.genome.ucsc.edu), 3,203 of 23,661 human genes registered in both the Refseq of the NCBI homepage and the UCSC homepage have isotypes, and the number of isotype mRNAs is 8,663. Therefore, a gene has about 2.7 isotypes on the average. The maximum number of isotypes in the gene is 23 (see Table 1). That is, it is shown that about 30% of human mRNAs has an alternatively spliced mRNA, which represents that the siRNA for a target mRNA should consider the alternative splicing mechanism.

TABLE 1

| Number of isotypes | Number of genes |
|---|---|
| 1 | 20,458 |
| 2 | 2,104 |
| 3 | 593 |
| 4 | 256 |
| 5 | 108 |
| 6 | 61 |
| 7 | 24 |
| 8 | 27 |
| 9 | 11 |
| 10 | 5 |
| 11 | 4 |
| 12 | 0 |
| 13 | 3 |
| 14 | 1 |
| 15 | 1 |
| 16 | 0 |
| 17 | 0 |
| 18 | 1 |
| 19 | 1 |
| 20 | 1 |
| 21 | 1 |
| 22 | 0 |
| 23 | 1 |
| Sum of genes | 23,661 |
| Sum of isotypes | 3,203 |
| Sum of mRNA isotypes | 8,663 |
| Average | 2.704652 |
| Maximum | 23 |

It has been reported that the expression pattern of the mRNA isotypes is different depending on the presence of diseases, tissue specificity and developmental stages (Sigalas I, Calvert A H, Anderson J J, Neal D E, Lunec J, Nat Med. 1996 August; 2(8):912-7; Weng M W, Lai J C, Hsu C P, Yu K Y, Chen C Y, Lin T S, Lai W W, Lee H, Ko J L, Environ Mol Mutagen. 2005 July; 46(1):1-11; Ando S, Sarlis N J, Krishnan J, Feng X, Refetoff S, Zhang M Q, Oldfield E H, Yen P M, Mol Endocrinol. 2001 September; 15(9):1529-38; Nakahata S, Kawamoto S, Nucleic Acids Res. 2005 Apr. 11; 33(7):2078-89; Lees-Murdock D J, Shovlin T C, Gardiner T, De Felici M, Walsh C P, Dev Dyn. 2005 April; 232(4):992-1002). The alternative spliced-variants have high base sequence homology among them. So, when the siRNA is designed in consideration of one specific isotype, it is difficult to find out proper siRNA candidates due to high homology among the isotypes.

Therefore, the present inventors has noticed that combination of intrinsic exons of each isotype can derive regions of exons, which specific target isotypes only have in common, and specific siRNA candidates of the regions, and we have developed a method of preparing siRNAs for selective inhibition of specific mRNA isotypes among various mRNA isotypes of a gene to inhibit effectively the expression of target mRNAs using the selected siRNAs.

### SUMMARY OF THE INVENTION

Various embodiments of the present invention are directed to provide a method for selecting siRNAs to selectively inhibit the expression of specific target mRNA isotypes among various mRNA isotypes of a gene and inhibiting effectively the expression of target mRNAs using the selected siRNAs.

### DETAILED DESCRIPTION OF THE PREFERRED EMBODIMENTS

In the specification, “isotype” refers to different types of mRNAs generated by alternative combinations of splicing from pre-mRNA generated by transcription of a gene.

“Exon” refers to regions in a sequence on genome DNA that is coding a gene representing the final mRNA sequence. In case of eukaryotes, one or more exons exist in a gene. Meanwhile, “intron” refers to regions existing between an exon and an adjacent exon thereof, and do not constitute mRNA sequences. The definition of these terms is well known to persons skilled in the art.

“Location information” refers to the information of locations of base sequences of exons on DNA corresponding to the mRNA sequences.

“Targe mRNA isotype” refers to one or more mRNAs which are objects for inhibiting the expression of mRNA isotypes of a gene, and “non-target mRNA isotype” refers to the rest mRNA isotypes except the target mRNA isotypes.

Hereinafter, the present invention will be described in detail.

A method of preparing siRNAs for selective inhibition of target mRNA isotypes of the present invention comprises the steps of:

(1) dividing target mRNA isotypes intended to inhibit the expression thereof and non-target mRNA isotypes from the mRNA isotypes of a gene;

(2) allotting a common location information region (A) of exons on genome DNA corresponding to the target mRNA isotypes;

(3) allotting a location information region (B) present specifically in exons of genome DNA corresponding to target mRNAs by excluding the common location information region of exons on the genome DNA corresponding to non-target mRNA from the location information region (A);

(4) determining base sequences in the target mRNAs corresponding to the location information region (B); and

(5) obtaining siRNA sequences for inhibiting the determined base sequences specifically.

In order to design siRNAs for inhibiting the expression of specific mRNA isotypes of a gene, base sequences existing specifically in the mRNA have to be determined. For example, a gene having three different mRNA isotypes is explained with reference to FIG. 1: the target mRNA of three isotypes consists of three exons, the rest two mRNA isotypes consist of three exons and two exons, respectively (represented by isotype 1 and isotype 2). In this case, a region existing specifically in the target mRNA corresponds to the region indicated by ▪ of the ┌mapping region┘ in the bottom of the figure. The region indicated by [symbol] represents a common region of the target mRNA and the mRNA isotype 1, and the region indicated by □ represents a common region of the target mRNA and the mRNA isotype 2. That is, it is possible to determine a region existing specifically in the target mRNA by excluding the regions overlapping with other non-target mRNA isotypes from the target mRNA region. When the target mRNA is two or more, the common region of the target mRNAs should be designated first. After excluding the overlapping regions of the rest (non-target) mRNA isotypes, desired regions can be determined.

To do this, information such as accession numbers of a gene or its mRNA isotypes, an mRNA base sequence, information on location of start and end points in exons of a genome, directional information of a gene in the genome is required. The information can be easily obtained through a proper database. The database can be obtained from the homepages of NCBI and of genome bioinformatics of UCSC and so on, and it is preferable to use the database in the homepages of NCBI and of genome bioinformatics of UCSC. These homepages are well-known to persons having ordinary skill in the art, and permits free access to all information required for the present invention. For example, files such as human.rna.fna, mouse.rna.fna, rat.rna.fna, human.rna.gbff, mouse.rna.gbff, rat.rna.gbff can be obtained from Refseq for providing several fields consisting of the “Gene” category of the NCBI homepage. In addition, tables such as RefGene and RefLink for providing MySQL database schema in ftp of the genome bioinformatics of USCS can be obtained from GoldenPath for all the organisms (human, rat and mouse). The table RefGene shows the detailed information on the number of exons in a gene and location information of the start and end points thereof.

Referring to A of FIG. 2, when a keyword such as a name of a gene to be inhibited and a mRNA accession number is inputted in the NCBI homepage, related data are displayed on a monitor, and it is possible to select mRNAs having the same gene name as that of the inputted gene. The selected mRNAs represent mRNA isotypes of the gene. The searched isotypes are divided into a target group and a non-target group to make a list. In designing siRNAs, the list can be used for two objects. First, the list can be used to select the siRNA designing region. Second, when a program such as BLAST is used to know for the homology of the designed siRNA with other non-target mRNAs and base sequences, the list can be used to reflect the comparison result of the rest (non-target) mRNA isotypes except the selected (target) isotypes.

The step (2) is explained with reference to the table shown in B and C of FIG. 2. Base sequences of the target mRNA can be searched in B of FIG. 2, and location information representing start and end points of exons on genome DNA of all isotypes searched by the step (1) can be searched in C of FIG. 2. Here, it is important that the location information of exons uses that of the genome DNA while the location information of base sequences uses that of the mRNAs. Although the base sequences of the genome DNA can be used directly in the designing of siRNAs, it has the following shortcomings. The changes of base sequences after the base sequences of the genome DNA are made into pre-mRNAs by transcription include the changes of base sequences by RNA editing as well as RNA splicing. It will be precise that information of base sequences takes not from the genome DNA but from the mRNA because the siRNA will target the mRNA after the changing process of the pre-mRNA has been completed. That is, although the method of selecting the designing region of siRNAs used in the present invention considers the alternative splicing, it also considers changes of other base sequences like the RNA editing.

The process of searching a region (A) which is a common region of target mRNAs can be performed preferably as follows:

(i) designating a start location as Sₜ and an end location as Eₜ for any exon of a genome DNA from 5′ end of the first target mRNA isotype, then the exon region is represented by (Sₜ, Eₜ). Designating a start location as Sᵢ and an end location as Eᵢ for any exon of a genome DNA from 5′ end of the other mRNA isotype intended to inhibit the expression with that of the first target mRNA isotype simultaneously, then the exon region is represented by (Sᵢ, Eᵢ)

(ii) confirming whether (Sₜ, Eₜ) and (Sᵢ, Eᵢ) have a common region. In case of Sᵢ≦Eₜ and Eᵢ≧Sₜ, two regions have a common region. This case belongs to one of four cases as shown in FIG. 4. The common region obtained from the four cases is as follows.

A. When Sₜ<Sᵢ and Eₜ>Eᵢ, then the common region is (Sᵢ, Eᵢ)

B. When Sₜ≧Sᵢ and Eₜ≦Eᵢ, then the common region is (Sₜ, Eₜ)

C. When Sₜ≧Sᵢ and Eₜ>Eᵢ, then the common region is (Sₜ, Eᵢ)

D. When Sₜ<Sᵢ and Eₜ≦Eᵢ, then the common region is (Sᵢ, Eₜ)

It is possible to obtain a common region intended to inhibit the expression of other target mRNA exons as well as the first target mRNA exons by the above-described process. If the same process is repeated after obtaining the above common region and the other common region with other target mRNA exons, it is possible to obtain a common region (hereinafter, referred to as a “region (A)”) of a genome DNA exon existing specifically in all mRNA isotypes for inhibition with the target mRNA isotypes. The region (A), a location information region which exists in common in combination of exons corresponding to all target mRNA isotypes, is represented by A=T₁∩T₂∩T₃∩ . . . ∩Tₙ, wherein “n” is a natural number representing the number of target mRNA isotypes intended to inhibit among mRNA isotypes of a gene, and Tₙ is a combination of coordinates for start and end locations of all exons on genome DNA corresponding to the n^{th} target mRNA, that is, Tₙ=(S_{Tn}, E_{Tn})₁, (S_{Tn}, E_{Tn})₂, . . . , (S_{Tn}, E_{Tn})ₓ. “x” represents the number of exons consisting of genome DNAs corresponding to target mRNA isotypes.

The step (3) is for selecting a region (B) which is a common region of exons existing only in target mRNA isotypes by excluding regions common to exon parts of a DNA of other mRNA isotypes from location information obtained from the step (2). Referring to FIG. 3, the step of selecting a designing region is performed using location information of start and end locations of exons in a genome DNA of target mRNAs and other mRNA isotypes obtained from the step (2) without other base sequence information. That is, a common region with other non-target mRNA isotypes is excluded from the common region of target mRNAs intended to inhibit their expression simultaneously, so as to select sequences which exist specifically in the desired target mRNA isotypes.

A method of excluding the common region with other non-target mRNA isotypes from the common region of the target mRNAs (or a region which remains after a common region of several isotypes is removed from the common region) is as follows:

(i) designating a start location as Sₜ and end location as Eₜ for any exon of a genome DNA from 5′ end of a target mRNA isotype (T₁), then the exon region is represented by (Sₜ, Eₜ). Designating a start location as Sᵢ and an end location as Eᵢ for i^{th} exon from a genome DNA from 5′ end of a non-target mRNA isotype (Q₁) required to remove the common region, then the exon region is represented by (Sᵢ, Eᵢ). First, of any (Sₜ, Eₜ) regions of genome DNA exons corresponding to target mRNAs, (Sₜ, Eₜ) which has a common region with any (Sᵢ, Eᵢ) regions satisfying Sᵢ≦Eₜ and Eᵢ≧Sₜ is selected. Here, if there are “n” numbers of (Sᵢ, Eᵢ) having a common region with (Sₜ, Eₜ), it is called (S₁, E₁)˜(Sₙ, Eₙ) from 5′ end, respectively.

(ii) If n=0, since there is no common region. Therefore, the whole region of (Sₜ, Eₜ) is preserved without any excluded parts. However, if n≧1, there is a common region. Therefore, it should be excluded. Referring to FIG. 5, the above process includes four cases. A region which remains after the common region is removed is represented as follows.

A. if Sₜ<S₁ and Eₜ>Eₙ, then the common region is (Sₜ, S₁), (E₁+1, S₂−1)˜(Eₙ₋₁+1, Sₙ−1), (Eₙ+1, Eₜ). If n=1 in this case, then the common region is (Sₜ, S₁), (E₁+1, Eₜ).

B. if Sₜ≧S₁ and Eₜ≦Eₙ, then the common region is (E₁+1, S₂−1)˜(Eₙ₋₁+1, Sₙ−1). If n=1, there is no common region.

C. if Sₜ≧S₁ and Eₜ>Eₙ, then the common region is (E₁+1, S₂−1)˜(Eₙ₋₁+1, Sₙ−1), (Eₙ+1, Eₜ). If n=1, then the common region is (E₁+1, Eₜ).

D. if Sₜ<S₁ and Eₜ≦Eₙ, then the common region is (Sₜ, S₁), (E₁+1, S₂−1)˜Eₙ₋₁+1, Sₙ−1). If n=1, then the common region is (Sₜ, S₁).

By using the method of FIG. 5, it is possible to obtain a region excluding the common region of the non-target mRNA isotype (Q₁) in a exon existing in the target mRNA isotype (T₁). Also, if the same process is repeated for the rest exons, it is possible to exclude a common region of other mRNA isotypes in the whole exons of the genome DNA corresponding to the target mRNA. If the common region with the region (A) is continuously removed from other non-target mRNA isotypes (Q₂, Q₃, . . . , Qₘ), it is possible to obtain location information (hereinafter, referred to a “regions (B)”) of the genome DNA exon existing specifically in the target mRNA. That is, the region (B) excluding a location information region which exists in common in combination of exons of non-target mRNA isotypes corresponding to all target mRNA isotypes is represented by B=A−(Q₁∪Q₂∪Q₃∪ . . . ∪Qₘ), wherein “m” is a natural number representing the number of non-target mRNA isotypes among mRNA isotypes of a gene, and Qₘ is a combination of coordinates for start and end locations of all exons on genome DNA corresponding to the m^{th} non-target mRNA, represented by Qₘ=(S_{Qm}, E_{Qm})₁, (S_{Qm}, E_{Qm})₂, . . . , (S_{Qm}, E_{Qm})ₓ. “x” is a natural number which represents the number of exons consisting of genome DNAs corresponding to non-target mRNA isotypes.

The step (4) is for converting location information of start and end points of the genome DNA exon selected in the step (3) into that of the target mRNA, and adding the location information to base sequences of the target mRNA of the step (1), thereby determining a base sequence for siRNA design. Referring to FIG. 6, there may be several exons in a gene. The exon is numbered 1 to x from 5′ end. The location information of bases is represented by S₁ to Sₓ where each exon starts, and by E₁ to Eₓ where each exon ends. The location information of a base in the genome of the k^{th} exon is represented by X_{G}. When the location information of the genome is a sense strand (or (+) strand) mRNA, if a any location information is X_{M+}, the relation between X_{G} and X_{M+} is represented by the following Equation 1.

$${X}_{M +} = ( {X}_{G} - {S}_{k} + 1 ) + {\sum }_{i = 1}^{k - 1} ( {E}_{i} - {S}_{i} + 1 ) \qquad [ Equation 1 ]$$

wherein k=1, then X_{M+}=(X_{G}−Sₖ+1)

Also, when the location information of the genome is a antisense strand (or (−) strand) mRNA, if a any location information is X_{M-}, the relation between X_{G} and X_{M-} is represented by the following Equation 2.

$$\begin{matrix}{X}_{M -} = ( mRNA length ) - {X}_{M +} + 1 \\ = {S}_{k} - {X}_{G} + {\sum }_{i = k}^{x} ( {E}_{i} - {S}_{i} + 1 )\end{matrix} \qquad [ Equation 2 ]$$

wherein k=1, then X_{M-}=Sₖ−X_{G}

By using the Equation 1 or Equation 2, the location information of (S₁, E₁)˜(Sₓ, Eₓ), the designing region selected in the genome DNA, can be converted into the location information of mRNAs, (Sₘ₁, Eₘ₁)˜(Sₘₓ, Eₘₓ). Base sequences corresponding to the location information converted in the base sequences of the target mRNA searched in the step (2) are used for designing siRNAs. When there are present certain consecutive regions, for example, (Sₘₖ, Eₘₖ) and (Sₘ₍ₖ₊₁₎, Eₘ₍ₖ₊₁₎) among the regions (Sₘ₁, Eₘ₁)˜(Sₘₓ, Eₘₓ), if Sₘ₍ₖ₊₁₎−Eₘₖ=1 is satisfied, the two regions are added to (Sₘₖ, Eₘ₍ₖ₊₁₎), and a base sequence suitable for the region is used in the designing of siRNAs.

The step (5) is for determining siRNA sequences for specifically inhibiting base sequences existing in common in the target mRNAs only. According to Tuschl rule etc. (S. M. Elbashir, J. Harborth, W. Lendeckel, A. Yalcin, Klaus Weber, T. Tuschl, Nature, 411, 494-498, 2001a; S. M. Elbashir, W. Lendeckel, T. Tuschl, Genes & Dev., 15, 188-200, 2001b; S. M. Elbashir, J. Martinez, A. Patkaniowska, W. Lendeckel, T. Tuschl, EMBO J., 20, 6877-6888, 2001c), siRNA sequences can be selected considering 3′ overhang types, GC content, repetition of specific bases, single nucleotide polymorphism (SNP) of base sequences, RNA secondary structures, and homology of non-target mRNA base sequences. In addition, binding energy types of double-stranded portions of siRNAs can be reflected in designing siRNA (Khvorova, A., Reynolds, A., Jayasena, S. D., Cell, 115(4), 505, 2003; Reynolds, A., Leake, D., Boese, Q., Scaringe, S., Marshall, W. S., Khvorova, A., Nat. Biotechnol., 22(3), 326-330, 2004). One of the representative examples to reflect the binding energy state to the siRNA design is that an energy difference between 5′ end and 3′ end is considered to predict siRNA efficiency. In this example, the binding position of the RNAi-induced silencing complex (RISC) affects the efficiency of siRNA depending on which one of two strands of siRNA the RISC bines to (Schwarz D S, Hutvagner G, Du T, Xu Z, Aronin N, Zamore P D., Cell, 115(2), 199-208, 2003). Preferably, ‘Method of Inhibiting Expression of Target mRNA Using siRNA Consisting of Nucleotide Sequence Complementary to Said Target mRNA’ (Korean Patent Application No. 2004-0103283 and PCT Patent Application No. PCT/KR2005/004207) developed by the present inventors can be used. The siRNA is a double strand (ds) RNA, and consists of 21-23 nucleotides, preferably 21 nucleotides, of which 19 nucleotides consist of a dsRNA part, and 1-3 nucleotides, preferable 2 nucleotides at both of the 3′-end consist of an overhang structure.

In order to confirm whether the steps (1) through (4) are properly performed, the location information of the target mRNA of the designed siRNA is selectively converted into the location information of the genome, and an additional step of confirming whether the designed siRNA inhibits the expression of the target mRNA may be performed.

The above step is actually performed in the reverse order of the step (4). If the siRNA is designed with the base sequence selected in the step (4), the location information of these designed siRNAs is equal to values such as X_{M+} or X_{M-} as location information of mRNAs. It is necessary to confirm on which exon of the genome siRNAs are located in order to identify whether the selection of the target mRNAs enables inhibition of the expression of the designed siRNAs. That is, the value of X_{M+} or X₋ is required to change into a value of X_{G}, and this is performed by the following Equation 3.

$$\begin{matrix}{X}_{G} = ( {X}_{M +} + {S}_{k} - 1 ) - {\sum }_{i = 1}^{k - 1} ( {E}_{i} - {S}_{i} + 1 ) \\ = {S}_{k} - {X}_{M -} + {\sum }_{i = k}^{x} ( {E}_{i} - {S}_{i} + 1 )\end{matrix} \qquad [ Equation 3 ]$$

wherein if k=1, then X_{G}=(X_{M+}+Sₖ−1)=Sₖ−X_{M-}.

In the Equation 3, since the value k is not a value after the siRNA designing is completed, the value k showing the exon in the genome the designed siRNA is located should be acquired using the given informations (X_{M+} or X_{M-}, and location information of the genome representing start and end points of exon). If the value k satisfying the condition of the Equation 4 is obtained, the value k to be used in the Equation 3 can be acquired.

$${\sum }_{i = 1}^{k - 1} ( {E}_{i} - {S}_{i} + 1 ) < ( {X}_{M +} {X}_{M -} ) \leq {\sum }_{i = 1}^{k} ( {E}_{i} - {S}_{i} + 1 ) \qquad [ Equation 4 ]$$

wherein if (X_{M+} [symbol] X_{M-})≦E₁−S₁+1, then k=1.

Furthermore, the present invention provides siRNAs prepared by the above-described method and a method of inhibiting the target mRNA isotypes only by introducing the siRNAs into mRNA isotypes of the gene. In this method, it is possible to effectively inhibit the expression of desired target mRNA isotypes only by introducing siRNAs selected through the above process to target mRNAs according to the conventional method.

### BRIEF DESCRIPTION OF THE DRAWINGS

FIG. 1 is a schematic diagram illustrating a method for selecting desired isotypes specifically in target mRNAs.

FIG. 2 is a table illustrating a part of databases including gene names, mRNA accession numbers of several isotypes corresponding to the names, mRNA base sequences, and location information on start and end points of exons of a genome.

FIG. 3 is a schematic diagram illustrating a method for obtaining a common region of target mRNAs to exclude a shared region of non-target isotypes in the common region.

FIG. 4 is a schematic diagram illustrating a method for obtaining a common region of target mRNAs with location information of the genome. The upper bar represented by (Sₜ, Eₜ) shows location information on start and end point of exons of a DNA corresponding to the first target mRNA. The central bar represented by (Sᵢ, Eᵢ) shows location information on start and end points of exons of a DNA corresponding to the second target mRNA. The lower bar shows location information on the common region of exons in the target mRNAs.

FIG. 5 is a schematic diagram illustrating a method for repeatedly removing a region of non-target isotypes in target mRNAs with location information of the genome DNA. The upper bar represented by (Sₜ, Eₜ) shows location information on start and end points of exons of a DNA corresponding to the target mRNA. The central bar represented by (S₁, E₁)˜(Sₙ, Eₙ) shows location information on start and end points of exons of a DNA corresponding to a mRNA isotype having a common region of the target mRNA. The lower bar shows location information on a residual region except a common region of isotype exons in the target mRNA exons.

FIG. 6 is a schematic diagram illustrating a method for converting location information of a genome into that of the target mRNA.

FIG. 7 is a schematic diagram illustrating information on isotypes expressed by alternative splicing of a MDM2 gene and protein domains consisting thereof.

FIG. 8 is a diagram illustrating locations of designed siRNAs in a genome about genes of Examples 1, 2 and 3 in a viewer using an siRNA design program based on exons.

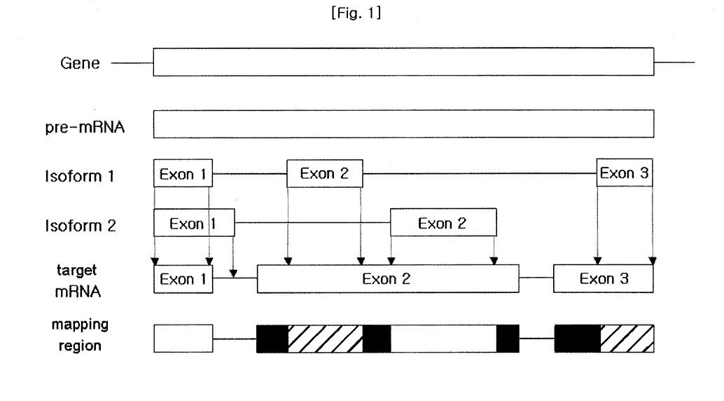

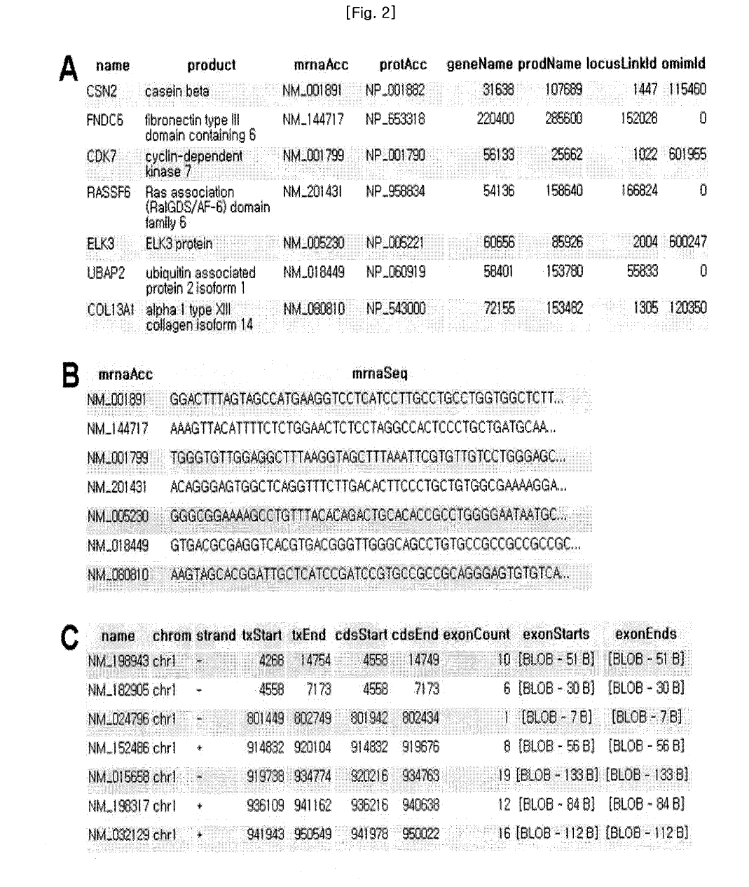

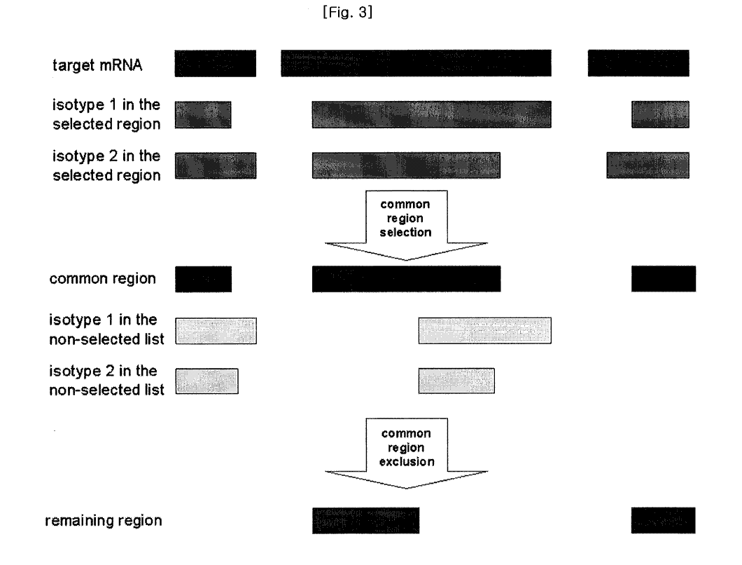

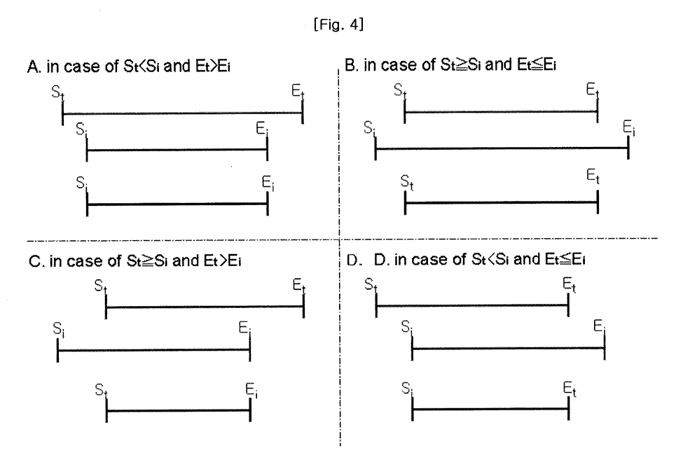

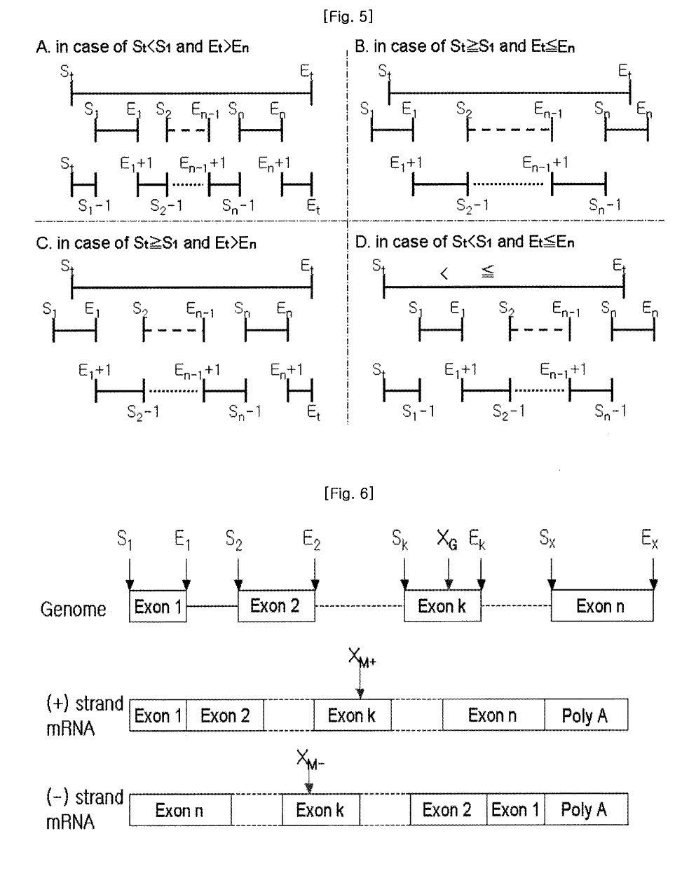

### EXAMPLES

The present invention will be described in detail with reference to examples below, which are not intended to be limiting.

### Example 1

### siRNA Design of MDM2 Gene-Usage in Disease Research

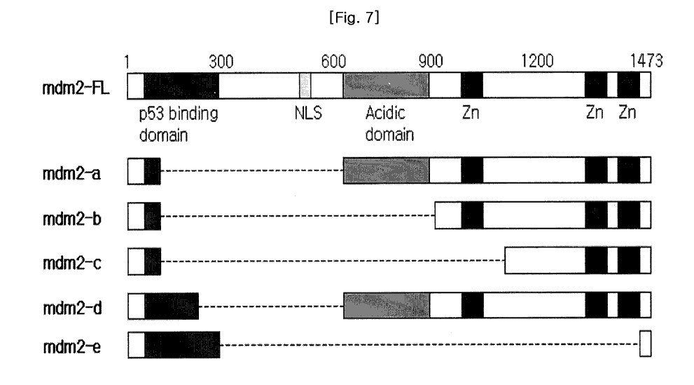

It has been reported that Human MDM2 (transformed 3T3 cell double minute 2, p53 binding protein) gene, which has 6 mRNA isotypes by alternative splicing, affects some types of cancer generation (ovarian cancer, bladder cancer) depending on the expression of the mRNA isotypes (Sigalas I, Calvert A H, Anderson J J, Neal D E, Lunec J, Nat Med. 1996 August; 2(8):912-7; Weng M W, Lai J C, Hsu C P, Yu K Y, Chen C Y, Lin T S, Lai W W, Lee H, Ko J L, Environ Mol Mutagen. 2005 July; 46(1):1-11; Ando S, Sarlis N J, Krishnan J, Feng X, Refetoff S, Zhang M Q, Oldfield E H, Yen P M, Mol Endocrinol. 2001 September; 15(9):1529-38). FIG. 7 shows information of the isotypes for MDM2 gene expressed by alternative splicing and the domains consisting thereof. In order to find out how the domain of the MDM2 gene affects cancer generation, siRNAs for inhibiting the expression of each domain selectively are designed as follows.

Of six isotypes shown in FIG. 7, isotypes having an acidic domain are NM_{—}002392, NM_{—}006878, NM_{—}006881. One of the best way to find out the relationship between the acidic domain and cancer generation is to inhibit the expression of the three isotypes selectively with siRNA only for the three isotypes, and to observe the changes of the cells. To do this, the present inventors designated the target mRNA as NM_{—}006881, and searched five isotypes NM_{—}002392, NM_{—}006878, NM_{—}006879, NM_{—}006880 and NM_{—}006882 in the NCBI homepage using the keyword of NM_{—}006881. Of these five isotypes, NM_{—}002392 and NM_{—}006878 were allotted in the list of target mRNA isotypes, and the rest three isotypes were allotted in the list of non-target mRNA isotypes.

After searching of mRNA base sequences of the target mRNA NM_{—}006881, the location information that represents start and end points of exons of the target mRNA and five mRNA isotypes were searched. The results are shown in Table 2. The location information of exons constitutes databases by parsing the RefGene table in Golden path of UCSC ftp. The UCSC ftp provides the RefGene table and schema.

TABLE 2

| mRNA accession | | |
|---|---|---|
| number | Start point of exons | End point of exons |
| NM_002392 | 67488247, 67489255, 67493601, 67496859, | 67488538, 67489339, 67493675, 67496922, |
| | 67500372, 67504410, 67504602, | 67500421, 67504477, 67504698, |
| | 67508818, 67515876, 67516719, | 67508978, 67516031, 67516796, |
| | 67519321 | 67520481 |
| NM_006878 | 67488247,67489255,67493601,67496859, | 67488538, 67489339, 67493675, 67496992, |
| | 67500372, 67504410, 67504602, | 67500421, 67504477, 67504698, |
| | 67519670 | 67520481 |
| NM_006879 | 67488247, 67489255, 67493601, 67496859, | 67488538, 67489339,67493675, 67496932, |
| | 67519865 | 67520481 |
| NM_006880 | 67488247, 67489255, 67493601, 67494650, | 67488538, 67489339, 67493675, 67494736, |
| | 67496859, 67519651 | 67496978, 67520481 |
| NM_006881 | 67488247, 67489255, 67493601, 67496859, | 67488538, 67489339, 67493675, 67496992, |
| | 67500372, 67504410, 67504602, | 67500421, 67504477, 67504627, |
| | 67519672 | 67520481 |
| NM_006882 | 67488247, 67489255, 67493601, 67496859, | 67488538, 67489339, 67493675, 67496978, |
| | 67519651 | 67520481 |

In the exon regions of the genome DNA of the first target mRNA NM_{—}002392, common regions of the second and third target mRNAs NM_{—}002392 and NM_{—}006878 were determined. The common regions were (67488247, 67488538), (67489255, 67489339), (67493601, 67493675), (67496859, 67496992), (67500372, 67500421), (67504410, 67504477), (67504602, 67504627) and (67519672, 67520481). In the above common regions, common regions with three non-target isotypes NM_{—}006879, NM_{—}006880 and NM_{—}006882 were excluded. The residual regions were (67496979, 67496992), (67500372, 67500421), (67504410, 67504477) and (67504602, 67504627). If the Equation 1 or Equation 2 is applied to location information of the four residual regions to convert location information of the mRNA, the results are (573, 586), (587, 636), (637, 704) and (705, 730). Since the above regions are adjacent to each other, one large region (573,730) is obtained by adding these regions together. If base sequences corresponding to the location information are identified in NM_{—}006881 sequence, it becomes to common base sequence present only in the desired mRNA isotypes. The base sequence is represented by SEQ. ID. NO: 1, and Table 3 shows some examples of siRNA sequences obtainable from the base sequence of SEQ. ID. NO: 1.

TABLE 3

| Start | | |
|---|---|---|
| location | siRNA base sequence | SEQ. ID. NO. |
| 692 | GAGTGATCAAAAGGACCTT | 2 |
| 691 | GGAGTGATCAAAAGGACCT | 3 |
| 698 | CCATGATCTACAGGAACTT | 4 |
| 684 | GAAGGTGGGAGTGATCAAA | 5 |
| 667 | AGAACAGGTGTCACCTTGA | 6 |
| 665 | TGAGAACAGGTGTCACCTT | 7 |
| 652 | GTACATCTGTGAGTGAGAA | 8 |
| 635 | GGAATCATCGGACTCAGGT | 9 |
| 695 | TGATCAAAAGGACCTTGTA | 10 |

When a program such as BLAST is used in order to consider homology of other mRNAs in the siRNA design, it is sufficient to consider the homology of other mRNA isotypes than the target mRNAs. Base sequences in Table 3 represent sense strand base sequences of 19-mer part consisting of a double strand in each siRNA. By the above-described method, it could be possible to design siRNAs for selectively inhibiting the expression of isotypes each having an acidic domain among several isotypes of MDM2 mRNAs. The siRNAs designed by the above method can be applied to a research for examining whether the relationship between cancer generation and the acidic domain of the MDM2 gene exists or not.

### Example 2

### siRNA Design for A2BP1 Gene-Usage in Research of Tissue Specificity

It has been reported that human A2BP1 (ataxin-2 binding protein 1) has four mRNA isotypes by alternative splicing, and the expression of the isotypes is affected by tissue specificity (Tabara H, Grishok A, Mello C C, Science, 282(5388), 430-1, 1998). In order to study tissue specific expression of the A2BP1 gene, it is preferable to inhibit the expression of one of the four isotypes only or to specifically inhibit the expression of isotypes showing specific functions or having specific domains, thereby observing the changes caused by the inhibition.

According to the NCBI homepage, A2BP1 has four isotypes such as NM_{—}018723, NM_{—}145891, NM_{—}145892, NM_{—}145893. Of these isotypes, the siRNA design for specifically inhibit the expression of NM_{—}018723 only can be performed as follows.

Using NM_{—}018723 as a keyword, the rest three isotypes NM_{—}145891, NM_{—}145892 and NM_{—}145893 were searched. After searching the mRNA sequence of the target mRNA isotype NM_{—}018723, the start and end points information (T₁) of exons of a DNA of the target mRNA and the location information (Q₁, Q₂, Q₃) representing start and end points of exons of DNA corresponding to three non-target mRNA isotypes were searched. The results are shown in Table 4. The location information of exons constitutes databases by parsing the RefGene table in Golden path of UCSC ftp.

TABLE 4

| mRNA accession | | |
|---|---|---|
| number | Start point of exons | End point of exons |
| NM_018723(T₁) | 6009133, 6306997, 6644605, 7042057, | 6009994, 6307059, 6644652, 7042100, |
| | 7508150, 7569780, 7577250, 7585552, | 7508392, 7569923, 7577303, 7585644, |
| | 7587374, 7597288, 7620606, | 7587434, 7597341, 7620686, 7643950, |
| | 7643818, 7654932, 7666777, 7699059, | 7654971, 7666841, 7699134, |
| | 7700626 | 7700847 |
| NM_145891(Q₁) | 7322752, 7508150, 7569780, 7577250, | 7323090, 7508392, 7569923, 7577303, |
| | 7585552, 7587374, 7597288, 7620606, | 7585644, 7587434, 7597341, 7620686, |
| | 7643818, 7661560, 7666777, | 7643950, 7661602, 7666841, 7699134, |
| | 7699059, 7700626 | 7702500 |
| NM_145892(Q₂) | 7322752, 7508150, 7569780, 7577250, | 7323090, 7508392, 7569923, 7577303, |
| | 7585552, 7587374, 7597288, 7620606, | 7585644, 7587434, 7597341, 7620686, |
| | 7643818, 7661560, 7666777, | 7643950, 7661602, 7666841, 7699134, |
| | 7699059, 7700704 | 7702500 |
| NM_145893(Q₃) | 7322752, 7508150, 7569780, 7577250, | 7323090, 7508392, 7569923, 757730 |
| | 7585552, 7587374, 7597288, 7620606, | 7585644, 7587434, 7597341, 7620686, |
| | 7643818, 7661560, 7666777, | 7643950, 7661602, 7666841, 7683370, |
| | 7683318, 7699059, 7700626 | 7699134, 7702500 |

As a result of excluding the common region of the non-target mRNA isotypes NM_{—}145891(Q₁), NM_{—}145892(Q₂), NM_{—}145893(Q₃) from the exon region (A) of the genome DNA of the target mRNA isotype NM_{—}018723(T₁), the residual region (B) becomes (6009133, 6009994), (6306997, 6307059), (6644605, 6644652), (7042059, 7042100) and (7654932, 7654971).). If the Equation 1 or Equation 2 is applied to location information of the four residual regions to convert location information (C) of the mRNA, C becomes (1, 861), (863, 925), (926, 973), (974, 1015) and (1879, 1918). Of these five region, (1, 861), (863, 925), (926, 973), (974, 1015) are adjacent to each other. If these regions are added together into one large region (1, 1015), the desired location information C′ becomes (1, 1015) and (1879, 1918). If base sequences corresponding to the location information are identified in NM_{—}018723 sequence, base sequences of regions for selectively inhibiting the expression of NM_{—}018723 only can be obtained. The base sequences are represented by SEQ. ID. NOs: 11 and 12, and Table 5 shows some examples of siRNA sequences obtainable from the base sequences of SEQ. ID. NO: 11 and 12.

TABLE 5

| Start | | |
|---|---|---|
| location | siRNA base sequence | SEQ. ID. NO |
| 987 | GAATTGTGAAAGAGAGCAG | 13 |
| 989 | TGTGAAAGAGAGCAGCT | 14 |

When a program such as BLAST is used in order to consider homology of the mRNA, it is sufficient to consider the homology of other mRNA isotypes than NM_{—}018723. Using the above-described method, it could be possible to design siRNAs for selectively inhibiting the expression of the isotypes where alternative splicing occurs with tissue-specific manner like A2BP1, and to examine tissue specificity of a gene such as A2BP1.

### Example 3

### siRNA Design of Dnmt3b Gene-Usage in Developmental Research

It has been reported that mouse Dnmt3b gene (DNA methyltransferase 3 B) has four isotypes by alternative splicing, which are directly involved in de novo methylation during developmental stages of mouse germ line cells, and splicing patterns of which are changing according to the developmental stages (Elbashir, S. M., Harborth, J., Lendeckel, W., Yalcin, A., Weber, K., Tuschl, T., Nature, 411, 494-498, 2001). For a research of the changes of the splicing pattern according to the developmental stages of the Dnmt3b gene, siRNAs are designed with reference to the following examples. The Dnmt3b gene has four isotypes NM_{—}001003960, NM_{—}001003961, NM_{—}001003963 and NM_{—}010068 by alternative splicing, and siRNA is designed with NM_{—}001003960 as the target mRNA. Using target mRNA NM_{—}001003960 as a keyword, the rest three non-target isotypes NM_{—}001003961, NM_{—}001003963, NM_{—}010068 were obtained. After confirming the mRNA sequence of target mRNA NM_{—}001003960, the location information that represents start and end points of exons of the target mRNA and three non-target mRNA isotypes were searched. The results are shown in Table 6. The location information of exons constitutes databases by parsing the RefGene table in Golden path of UCSC ftp.

TABLE 6

| mRNA | | |
|---|---|---|
| accession | | |
| number | Start point of exons | End point of exons |
| NM_001003960 | 153106394, 153107722, 153118362, | 153106677, 153107836, 153118539, |
| | 153119080, 153119651, 153122189, | 153119141, 153119770, 153122314, |
| | 153122844, 153124441, 153126685, | 153123038, 153124599, 153126792, |
| | 153127240, 153129153, 153129433, | 153127384, 153129272, 153129477, |
| | 153130924, 153131286, 153131795, | 153131003, 153131398, 153131981, |
| | 153132666, 153133633, 153134108, | 153132750, 153133778, 153134198, |
| | 153134448, 153135408, 153140545, | 153134596, 153135493, 153140614, |
| | 153141296, 153143195 | 153141414, 153144663 |
| NM_001003961 | 153106394, 153107722, 153118362, | 153106677, 153107836, 153118539, |
| | 153119080, 153119651, 153122189, | 153119141, 153119770, 153122314, |
| | 153122844, 153124441, 153126685, | 153123038, 153124599, 153126792, |
| | 153127240, 153127786, 153129153, | 153127384, 153127845, 153129272, |
| | 153129433, 153130924, 153131286, | 153129477, 153131003, 153131398, |
| | 153131795, 153132666, 153133633, | 153131981, 153132750, 153133778, |
| | 153134108, 153134448, 153135408, | 153134198, 153134596, 153135493, |
| | 153140545, 153141296, 153143195 | 153140614, 153141414, 153144663 |
| NM_001003963 | 153106394, 153107722, 153118362, | 153106677, 153107836, 153118539, |
| | 153119080, 153119651, 153122189, | 153119141, 153119770, 153122314, |
| | 153122844, 153124441, 153126685, | 153123038, 153124599, 153126792, |
| | 153127240, 153127786, 153129153, | 153127384, 153127845, 153129272, |
| | 153129433, 153130924, 153131286, | 153129477, 153131003, 153131398, |
| | 153131795, 153132666, 153133633, | 153131981, 153132750, 153133778, |
| | 153134108, 153134448, 153135408, | 153134198, 153134596, 153135493, |
| | 153143195 | 153144663 |
| NM_010068 | 153106394, 153107722, 153118362, | 153106677, 153107836, 153118539, |
| | 153119080, 153119651, 153122189, | 153119141, 153119770, 153122314, |
| | 153122844, 153124441, 153126685, | 153123038, 153124599, 153126792, |
| | 153127240, 153129153, 153129433, | 153127384, 153129272, 153129477, |
| | 153130924, 153131286, 153131795, | 153131003, 153131398, 153131981, |
| | 153132666, 153133633, 153134108, | 153132750, 153133778, 153134198, |
| | 153134448, 153135408, 153143195, | 153134596, 153135493, 153144663 |

After obtaining common exon region (A) of the genome of the target mRNAs NM_{—}001003960 (T₁) and NM_{—}001003961 (T2) (i.e. (A)=T₁∩T₂), the common region of the non-target isotypes of NM_{—}001003963(Q₁), NM_{—}010068(Q₂) (i.e. (Q₁∪Q₂)) was excluded from the common region of the target mRNA (A). The residual regions (B=A−(Q₁∪Q₂)) obtained from the above process become (153140545, 153140614) and (153141296, 153141414). These are the regions where it is possible to design siRNAs for inhibiting the expression of both NM_{—}001003960(T1) and NM_{—}001003961(T₂) simultaneously. If the Equation 1 or Equation 2 is applied to location information of the two residual regions to convert location information of the mRNA, C becomes (2595, 2664) and (2665, 2783). Since the two regions are adjacent to each other, one large region (C′) (2595, 2783) is obtained by adding these regions together. Using the region (C′), desired siRNAs can be designed. The base sequence of the target mRNA NM_{—}001003960(T₁) corresponding to this location information can be a base sequence for selectively inhibiting the expression of NM_{—}001003960(T₁) and NM_{—}001003961(T₂). The base sequence of the region is represented by SEQ. ID. NO: 15, and Table 7 shows some examples of siRNAs obtainable from the base sequence of SEQ. ID. NO: 15.

TABLE 7

| Start | | |
|---|---|---|
| location | siRNA | SEQ. ID. NO. |
| 2644 | TGCCTGGAGTTCAGTAGGACAGC | 16 |
| 2698 | AAGTCGAACTCCATCAGACAGGG | 17 |
| 2758 | GACGACGTTTTGTGGTGCACTGA | 18 |
| 2642 | ACTGCCTGGAGTTCAGTAGGACA | 19 |
| 2704 | AACTCCATCAGACAGGGCAAAAA | 20 |
| 2726 | ACCAGCTTTTCCCTGTAGTCATG | 21 |
| 2653 | TTCAGTAGGACAGCAAAGTTAAA | 22 |
| 2613 | TTCAAAGAATGATAAGCTCGAGC | 23 |
| 2697 | CAAGTCGAACTCCATCAGACAGG | 24 |

When a program such as BLAST is used in order to consider homology of other mRNAs in the siRNA design, it is sufficient to consider the homology of other mRNA isotypes than the target mRNA isotypes NM_{—}001003960(T₁) and NM_{—}001003961(T₂). The siRNAs designed by the above-described method can be used in researches for a splicing process of Dnmt3b gene in a mouse germ line cell and for possible changes according to the developmental stages.

### Example 4

### Development of siRNA Designing Program Considering Alternative Splicing

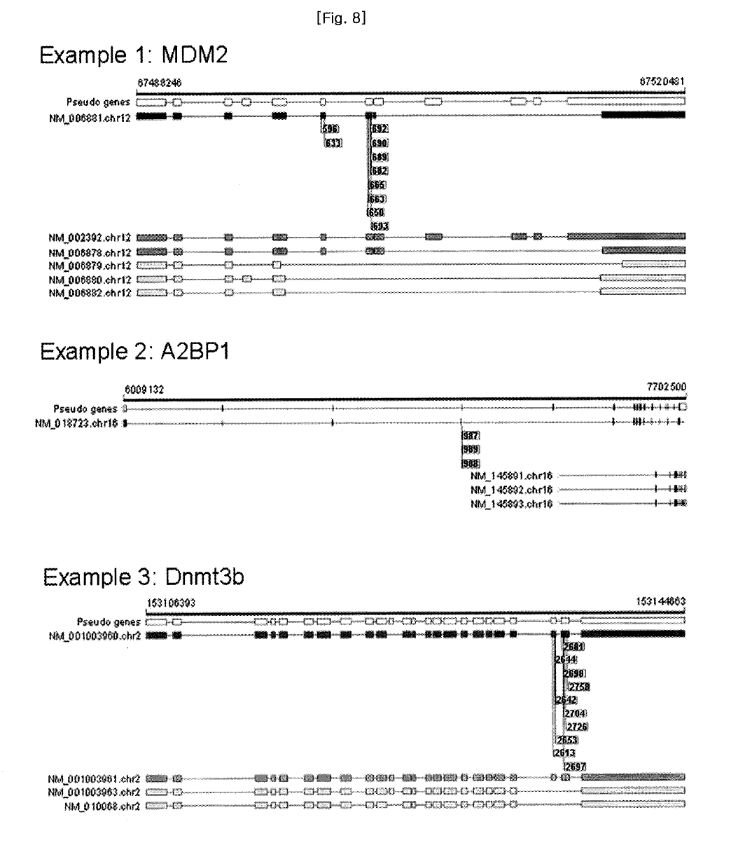

The present inventors tried to develop a novel siRNA designing program encompassing ‘a method of selecting an siRNA designing region considering alternative splicing’ that is the content of the present invention and ‘Method of Inhibiting Expression of Target mRNA Using siRNA Consisting of Nucleotide Sequence complementary to Said Target mRNA’ that the present inventors has filed previously. The program selects a target region for specific isotypes using the former method, and determines highly efficient sequences by the latter method. The program enables website services. By accessing the website, a user can search information easily on target mRNAs and isotypes, select isotypes for inhibiting the expression thereof, and automatically design highly efficient siRNAs targeting the selected isotypes. The program can provide an implicit viewer so that a user can perform a design process while identifying exon distribution of each isotype. FIG. 8 shows a viewer part of the monitor as a result of siRNA design of three genes of Examples 1, 2 and 3, where a red track represents target mRNAs, a green track represents an isotypes of the selected list, a yellow track represents an isotypes of the unselected list, and a tag indicated by numbers represents a location information of the designed siRNA. The number indicated in the tag shows location information of the mRNA of the siRNA, which is converted into location information of the genome by the step (e) and displayed in a location shown in the viewer. As shown in results, the designed siRNAs are precisely designed in an exon region that target mRNAs and isotypes of the selected list have in common.

### Example 5

### Preparation of Exon Database and siRNA Library for Splicing Products

As shown in the above examples, steps of selecting a target mRNA, selecting location information of exons shared by specific isotypes only, and designing siRNAs are automated in the present invention. As the other application plan of the automated modules, the entire human (or mouse, rat) genes may be arranged to produce a new secondary database.

For the secondary database, after preparing combination of splicing products of a specific gene, and location information of start and end points of exons on a genome shared by the isotypes in the combination is obtained using the automated modules and then databased. If the database is represented by a FASTA file provided in the NCBI homepage, the result can be as follows.
>(biological species)|(gene name)|(targeted isotype list)|(non-target isotype list) (start of exon 1):(end of exon 1), (start of exon 2):(end of exon 2), . . . .

The following result shows location information of the shared exons using the automated modules in all combinations of the splicing products.

>mouse|Dnmt3b|NM_001003960, |NM_001003961, NM_001003963, NM_010068,
>mouse|Dnmt3b|NM_001003961, |NM_001003960, NM_001003963, NM_010068,
>mouse|Dnmt3b|NM_001003963, |NM_001003960, NM_001003961, NM_010068,
>mouse|Dnmt3b|NM_010068, |NM_001003960, NM_001003961, NM_001003963,
>mouse|Dnmt3b|NM_001003960, NM_001003961, |NM_001003963, NM_010068,
153140545:153140614, 153141296:153141414,
>mouse|Dnmt3b|NM_001003960, NM_001003963, |NM_001003961, NM_010068,
>mouse|Dnmt3b|NM_001003960, NM_010068, |NM_001003961, NM_001003963,
>mouse|Dnmt3b|NM_001003961, NM_001003963, |NM_001003960, NM_010068,
153127786:153127845,
>mouse|Dnmt3b|NM_001003961, NM_010068, |NM_001003960, NM_001003963,
>mouse|Dnmt3b|NM_001003963, NM_010068, |NM_001003960, NM_001003961,
>mouse|Dnmt3b|NM_001003960, NM_001003961, NM_001003963, |NM_010068,
>mouse|Dnmt3b|NM_001003960, NM_001003961, NM_010068, |NM_001003963,
>mouse|Dnmt3b|NM_001003960, NM_001003963, NM_010068, |NM_001003961,
>mouse|Dnmt3b|NM_001003961, NM_001003963, NM_010068, |NM_001003960,
>mouse|Dnmt3b|NM_001003960, NM_001003961, NM_001003963, NM_010068,|
153106394:153106677, 153107722:153107836, 153118362:153118539, 153119080:153119141, 153119651:153119770,
153122189:153122314, 153122844:153123038, 153124441:153124599, 153126685:153126792,
153127240:153127384, 153129153:153129272, 153129433:153129477, 153130924:153131003, 153131286:153131398,
153131795:153131981, 153132666:153132750, 153133633:153133778, 153134108:153134198, 153134448:153134596,
153135408:153135493, 153143195:153144663,

The exon location information of the genome can be replaced with other information including the following base sequence information.
>(biological species)|(gene name)|(targeted isotype list)|(non-target isotype list) (base sequence of exon 1):(base sequence of exon 2), . . . .

To make this type of database using classification for combinations of the splicing products is valuable in an industrial aspect. If the library (database) is previously prepared and synthesized for sale according to the classification, time and cost are reduced compared to a conventional system for synthesizing a library after taking an order from a user. The following database shows a partial content of the siRNA library prepared by the automated modules and combination of splicing products.

>human|PTPDC1|NM 152422,|NM_177995,
human, PTPDC1, NM_152422, 4398, 270, ATGCAGAGGGAAACCCAACTTTC, 0.53, false, 15, 72.5, 81.6
human, PTPDC1, NM_152422, 4398, 271, TGCAGAGGGAAACCCAACTTTCC, 0.47, false, 15, 50.0, 67.3
human, PTPDC1, NM_152422, 4398, 142, CTCCACCTCAGACCCAGTACTGC, 0.58, false, 14, 62.9, 66.8
human, PTPDC1, NM_152422, 4398, 210, CCACGAAGCTGCTGTCCTCGTCC, 0.58, false, 15, 72.5, 61.3
human, PTPDC1, NM_152422, 4398, 250, GGCTGTTTCCTCAGTCAGCCATG, 0.53, false, 14, 40.4, 59.3
human, PTPDC1, NM_152422, 4398, 256, TTCCTCAGTCAGCCATGCAGAGG, 0.58, false, 15, 50.0, 57.6
human, PTPDC1, NM_152422, 4398, 266, AGCCATGCAGAGGGAAACCCAAC, 0.58, false, 15, 50.0, 57.6
human, PTPDC1, NM_152422, 4398, 254, GTTTCCTCAGTCAGCCATGCAGA, 0.53, false. 15, 50.0, 55.8
human, PTPDC1, NM_152422, 4398, 269, CATGCAGAGGGAAACCCAACTTT, 0.53, false. 15, 50.0, 55.8
human, PTPDC1, NM_152422, 4398, 291, TCCCCGAAAGAAAAAGAAATTTA, 0.32, false. 15, 40.4, 54.5
>human|PTPDC1|NM_177995,|NM_152422,
human, PTPDC1, NM_177995, 4515, 399, GCGAGTGTGTTGCAAACATGAAA, 0.42, false, 15, 71.3, 79.8
human, PTPDC1, NM_177995, 4515, 349, AGGAGTCTTGCCTCAGAATGAAC, 0.47, false, 15, 61.8, 74.8
human, PTPDC1, NM_177995, 4515, 373, ACCATATTCTACCTTGGTGAATC, 0.37, false, 14, 62.9, 74.5
human, PTPDC1, NM_177995, 4515, 389, GTGAATAACAGCGAGTGTGTTGC, 0.42, false, 13, 62.9, 74.5
human, PTPDC1, NM_177995, 4515, 386, TTGGTGAATAACAGCGAGTGTGT, 0.47, false, 13, 59.6, 72.4
human, PTPDC1, NM_177995, 4515, 372, AACCATATTCTACCTTGGTGAAT, 0.42, false, 14, 59.6, 71.4
human, PTPDC1, NM_177995, 4515, 350, GGAGTCTTGCCTCAGAATGAACA, 0.42, false, 15, 71.3, 70.3
human, PTPDC1, NM_177995, 4515, 400, CGAGTGTGTTGCAAACATGAAAG, 0.37, false, 15, 71.3, 70.3
human, PTPDC1, NM_177995, 4515, 362, CAGAATGAACAACCATATTCTAC, 0.32, false. 15, 62.9, 67.8
human, PTPDC1, NM_177995, 4515, 378, ATTCTACCTTGGTGAATAACAGC, 0.37, false, 14, 62.9, 64.9
>human|PTPDC1|NM_152422, NM_177995,|
human, PTPDC1, NM_152422, 4398, 1132, GCCAGTGATGATGAAGGATGTGT, 0.42, false, 15, 90.4, 91.9
human, PTPDC1, NM_152422, 4398, 926, CTCTGTGTAAGGGAATTTACTCA, 0.37, false, 14, 80.9, 86.9
human, PTPDC1, NM_152422, 4398, 980, TGCTGTGATCCCAAAGCACATGC, 0.47, false, 15, 80.9, 85.9
human, PTPDC1, NM_152422, 4398, 1154, TCCGAAGGACCTGGTCTCTCTGC, 0.58, false, 15, 90.4, 84.2
human, PTPDC1, NM_152422, 4398, 370, TGGACACATGGCATGTTCCATGG, 0.47, false, 14, 77.5, 83.8
human, PTPDC1, NM_152422, 4398, 716, GCGTCTCTTACTACTATCCTAGA, 0.37, false, 15, 77.5, 83.8
human, PTPDC1, NM_152422, 4398, 718, GTCTCTTACTACTATCCTAGATA, O.37, false, 15, 77.5, 83.8
human, PTPDC1, NM_152422, 4398, 1793, CACTGTCAGTGTAAAACTCATGG, 0.37, false, 14, 77.5, 83.8
human, PTPDC1, NM_152422, 4398, 1123, GGAGAACAGGCCAGTGATGATGA, 0.47, false, 15, 90.4, 82.4
human, PTPDC1, NM_152422, 4398, 270, ATGCAGAGGGAAACCCAACTTTC, 0.53, false, 15, 72.5, 81.6

### INDUSTRIAL APPLICABILITY

As described above, according to the method of the present invention, siRNAs for selectively inhibiting the expression of specific target mRNA isotypes in a gene where several isotypes exist by alternative splicing can be easily prepared. The method enables siRNA design for genome-wide genes, which can be used as a good tool for functional genomics. The siRNA libraries prepared through automated modules can reduce cost and time in an industrial aspect, and contribute localization of them interworking with an siRNA synthesis and sale system.

### SEQUENCE LISTING

Sequence listing is attached herewith.

1-12. (canceled)

13. A method for preparing siRNAs to selectively inhibit target mRNA isotypes, the method comprising:
(1) dividing the target mRNA isotypes intended to inhibit the expression thereof and non-target mRNA isotypes from the mRNA isotypes of a gene;
(2) allotting a common location information region (A) of exons on genome DNA corresponding to the target mRNA isotypes;
(3) allotting a location information region (B) present specifically in exons of genome DNA corresponding to target mRNAs by excluding the location information region of exons on genome DNA corresponding to non-target mRNA from the location information region (A);
(4) determining base sequences in the target mRNAs corresponding to the location information region (B); and
(5) obtaining siRNA sequences for inhibiting the determined base sequences specifically.

14. The method according to claim 13, wherein A=T₁∩T₂∩T₃∩ . . . ∩Tₙ, B=A−(Q₁∪Q₂∪Q₃∪ . . . ∪Qₘ),
wherein “n” is a natural number representing the number of target mRNA isotypes intended to inhibit among mRNA isotypes of a gene,
“m” is a natural number representing the number of non-target mRNA isotypes among mRNA isotypes of a gene,
“Tₙ” is a combination of coordinates for start and end locations of all exons on genome DNA corresponding to the n^{th} target mRNA, and
“Qₘ” is a combination of coordinates for start and end locations of all exons on genome DNA corresponding to the m^{th} non-target mRNA.

15. The method according to claim 13, wherein in the determination of the base sequences in the target mRNA in step (4), when the location information of the genome DNA is a sense strand mRNA, the target mRNA consists of “x” exons on genome DNA, the location information of bases is represented by S₁ to Sₓ where each exon starts, and by E₁ to Eₓ where each exon ends, and the location information of a base in the genome of the k^{th} exon is represented by X_{G}, then X_{M+} on the target mRNA corresponding to X_{G} is obtained by the following Equation 1:
$${X}_{M +} = ( {X}_{G} - {S}_{k} + 1 ) + {\sum }_{i = 1}^{k - 1} ( {E}_{i} - {S}_{i} + 1 ) \qquad [ Equation 1 ]$$
wherein k=1, then X_{M+}=(X_{G}−Sₖ+1).

16. The method according to claim 13, wherein in the determination of the base sequences in the target mRNA in step (4), when the location information of the genome DNA is a antisense strand mRNA, the target mRNA consists of “x” exons on genome DNA, the location information of bases is represented by S₁ to Sₓ where each exon starts, and by E₁ to Eₓ where each exon ends, the location information of a base in the genome of the k^{th} exon is represented by X_{G}, and Xₘ₊ represents location information on the target mRNA corresponding to X_{G} when the location information of the genome DNA is a sense strand mRNA, then X_{M-} on the target mRNA corresponding to X_{G} is obtained by the following Equation 2:
$$\begin{matrix}{X}_{M -} = ( mRNA length ) - {X}_{M +} + 1 \\ = {S}_{k} - {X}_{G} + {\sum }_{i = k}^{n} ( {E}_{i} - {S}_{i} + 1 )\end{matrix} \qquad [ Equation 2 ]$$
wherein k=1, then X_{M-}=Sₖ−X_{G}.

17. The method according to claim 13, wherein the siRNA sequence of the step (5) is obtained by considering one or more factors selected from the group consisting of 3′ overhang type, GC content, repetition of specific bases, SNP of base sequence, RNA secondary structure, and homology of a non-target mRNA base sequences.

18. The method according to claim 13, wherein the siRNA in step (5) is determined in consideration of binding energy differences of 5′ end and 3′ end of the mRNA.

19. The method according to claim 13, further comprising the step of converting the location information of the target mRNA (X_{M+} or X_{M-}) into the location information of the genome (X_{G}) to verify whether an siRNA inhibits the expression of the target mRNA after the step (4), wherein Xₘ₊ represents location information on the target mRNA corresponding to X_{G} when the location information of the genome DNA is a sense strand mRNA, and Xₘ₋ represents location information on the target mRNA corresponding to X_{G} when the location information of the genome DNA is an antisense strand mRNA.

20. The method according to claim 19, wherein the converting step is performed by the following Equations 3 and 4, when the target mRNA consists of “x” exons on genome DNA, the location information of bases is represented by S₁ to Sₓ where each exon starts, and by E₁ to Eₓ where each exon ends, the location information of a base in the genome of the k^{th} exon is represented by X_{G}, Xₘ₊ represents location information on the target mRNA corresponding to X_{G} when the location information of the genome DNA is a sense strand mRNA, and Xₘ₋ represents location information on the target mRNA corresponding to X_{G} when the location information of the genome DNA is an antisense strand mRNA:
$$\begin{matrix}{X}_{G} = ( {X}_{M +} + {S}_{k} - 1 ) - {\sum }_{i = 1}^{k - 1} ( {E}_{i} - {S}_{i} + 1 ) \\ = {S}_{k} - {X}_{M -} + {\sum }_{i = k}^{n} ( {E}_{i} - {S}_{i} + 1 )\end{matrix} \qquad [ Equation 3 ]$$
wherein k=1, then X_{G}=(X_{M+}+Sₖ−1)−Sₖ−X_{M-}
$${\sum }_{i = 1}^{k - 1} ( {E}_{i} - {S}_{i} + 1 ) < ( {X}_{M +} {X}_{M -} ) \leq {\sum }_{i = 1}^{k} ( {E}_{i} - {S}_{i} + 1 ) \qquad [ Equation 4 ]$$
wherein if (X_{M+} [symbol] X_{M-})≦E₁−S₁+1, then k=1.

21. The method according to claim 13, wherein the siRNA is a double strand RNA having 21 nucleotides.

22. The method according to claim 13, wherein the siRNA has a dsRNA portion of 19 nucleotide length and an overhang structure of 1˜3 nucleotides at both 3′-ends.

23. An siRNA prepared by one of the method according to claim 13.

24. A method for selectively inhibiting the expression of target mRNA isotypes by introducing the siRNA of claim 23 into mRNA isotypes of a target gene.


SEQUENCE LISTING (61 sequences)

SEQ ID NO: 1 - DNA, 158 bp, Homo sapiens
gtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaaga

SEQ ID NO: 2 - DNA, 19 bp, Artificial Sequence
  siRNA sequence for alternatively spliced human MDM2 gene
gagtgatcaaaaggacctt

SEQ ID NO: 3 - DNA, 19 bp, Artificial Sequence
  siRNA sequence for alternatively spliced human MDM2 gene
ggagtgatcaaaaggacct

SEQ ID NO: 4 - DNA, 19 bp, Artificial Sequence
  siRNA sequence for alternatively spliced human MDM2 gene
ccatgatctacaggaactt

SEQ ID NO: 5 - DNA, 19 bp, Artificial Sequence
  siRNA sequence for alternatively spliced human MDM2 gene
gaaggtgggagtgatcaaa

SEQ ID NO: 6 - DNA, 19 bp, Artificial Sequence
  siRNA sequence for alternatively spliced human MDM2 gene
agaacaggtgtcaccttga

SEQ ID NO: 7 - DNA, 19 bp, Artificial Sequence
  siRNA sequence for alternatively spliced human MDM2 gene
tgagaacaggtgtcacctt

SEQ ID NO: 8 - DNA, 19 bp, Artificial Sequence
  siRNA sequence for alternatively spliced human MDM2 gene
gtacatctgtgagtgagaa

SEQ ID NO: 9 - DNA, 19 bp, Artificial Sequence
  siRNA sequence for alternatively spliced human MDM2 gene
ggaatcatcggactcaggt

SEQ ID NO: 10 - DNA, 19 bp, Artificial Sequence
  siRNA sequence for alternatively spliced human MDM2 gene
tgatcaaaaggaccttgta

SEQ ID NO: 11 - DNA, 1015 bp, Homo sapiens
tccggctctcctttgtttattttctaatctatatttttactggaagatttcctctttattctctcccgccctcctacaagcgctcttgctggccgtctgggtgcacacaccgctccctcgatcaccccagcccccttcctggtctcccgagcgcggggtttgaaggtcacctcctttccagtccccgtgcgagccgcgctgccgccgcctcctccagccagagtcggtgggactggctgcgctgccctgaagtggttctccaagcagcgcggagggtggcggacggcggacggagcccaggggccgcgtcgggtggggaaacccgaactcgcggaggggaatccctcccccttcgccccagccccccagcagcacccgcggtggggcgggggcgctctgccagccccgggaacagcagaggcggcggcactggctggacccacgcgcgcgcctccggggctgaagaaggaaggagtgagccgagccgagcaccccacatctggaggggacagccagccgtgggccccgccccggcgtccggagcaggagaattccgagcttcttgcccaggcagagagagcaggagcggaccgcgcgcccgggattgagagtccttgcgctccagacccccacccagtggccgccaggtcccgcctgtccggaccctcgccgcgcccaggcaggcgcgccagggcggggctgacctgcccgcgaagttgcggacagtgcgtgagaaaccagcacccccttccgccgcctccagcttatggtgagtgtggctgggggtgcagagagcgcacgggaattcgggggtctggggccgagaacgtgaccgcagccgggctcgccgggagttctaggaaactggtcaaagaactcatgcaagtggaacttacagcttccttgatcggactcagcattcagatatcaaagcagactgcaatacctgcgtggaaatagaagacagaaaggtttcaagacaacagatgaattgtgaaagagagcagctaagggg

SEQ ID NO: 12 - DNA, 40 bp, Homo sapiens
ttgtttaccaggatggattttatggtgcagacatttatgg

SEQ ID NO: 13 - DNA, 19 bp, Artificial Sequence
  siRNA sequence for alternatively spliced human A2BP1 gene
gaattgtgaaagagagcag

SEQ ID NO: 14 - DNA, 19 bp, Artificial Sequence
  siRNA sequence for alternatively spliced human A2BP1 gene
attgtgaaagagagcagct

SEQ ID NO: 15 - DNA, 189 bp, Mus musculus
gaacaggcccgtgatggcttcaaagaatgataagctcgagctgcaggactgcctggagttcagtaggacagcaaagttaaagaaagtgcagacaataaccaccaagtcgaactccatcagacagggcaaaaaccagcttttccctgtagtcatgaatggcaaggacgacgttttgtggtgcactgagct

SEQ ID NO: 16 - DNA, 23 bp, Artificial Sequence
  siRNA sequence for alternatively spliced mouse Dnmt3b gene
tgcctggagttcagtaggacagc

SEQ ID NO: 17 - DNA, 23 bp, Artificial Sequence
  siRNA sequence for alternatively spliced mouse Dnmt3b gene
aagtcgaactccatcagacaggg

SEQ ID NO: 18 - DNA, 23 bp, Artificial Sequence
  siRNA sequence for alternatively spliced mouse Dnmt3b gene
gacgacgttttgtggtgcactga

SEQ ID NO: 19 - DNA, 23 bp, Artificial Sequence
  siRNA sequence for alternatively spliced mouse Dnmt3b gene
actgcctggagttcagtaggaca

SEQ ID NO: 20 - DNA, 23 bp, Artificial Sequence
  siRNA sequence for alternatively spliced mouse Dnmt3b gene
aactccatcagacagggcaaaaa

SEQ ID NO: 21 - DNA, 23 bp, Artificial Sequence
  siRNA sequence for alternatively spliced mouse Dnmt3b gene
accagcttttccctgtagtcatg

SEQ ID NO: 22 - DNA, 23 bp, Artificial Sequence
  siRNA sequence for alternatively spliced mouse Dnmt3b gene
ttcagtaggacagcaaagttaaa

SEQ ID NO: 23 - DNA, 23 bp, Artificial Sequence
  siRNA sequence for alternatively spliced mouse Dnmt3b gene
ttcaaagaatgataagctcgagc

SEQ ID NO: 24 - DNA, 23 bp, Artificial Sequence
  siRNA sequence for alternatively spliced mouse Dnmt3b gene
caagtcgaactccatcagacagg

SEQ ID NO: 25 - DNA, 50 bp, Homo sapiens
ggactttagtagccatgaaggtcctcatccttgcctgcctggtggctctt

SEQ ID NO: 26 - DNA, 50 bp, Homo sapiens
aaagttacattttctctggaactctcctaggccactccctgctgatgcaa

SEQ ID NO: 27 - DNA, 50 bp, Homo sapiens
tgggtgttggaggctttaaggtagctttaaattcgtgttgtcctgggagc

SEQ ID NO: 28 - DNA, 50 bp, Homo sapiens
acagggagtggctcaggtttcttgacacttccctgctgtggcgaaaagga

SEQ ID NO: 29 - DNA, 50 bp, Homo sapiens
gggcggaaaagcctgtttacacagactgcacaccgcctggggaataatgc

SEQ ID NO: 30 - DNA, 50 bp, Homo sapiens
gtgacgcgaggtcacgtgacgggttgggcagcctgtgccgccgccgccgc

SEQ ID NO: 31 - DNA, 50 bp, Homo sapiens
aagtagcacggattgctcatccgatccgtgccgccgcagggagtgtgtca

SEQ ID NO: 32 - DNA, 23 bp, Homo sapiens
atgcagagggaaacccaactttc

SEQ ID NO: 33 - DNA, 23 bp, Homo sapiens
tgcagagggaaacccaactttcc

SEQ ID NO: 34 - DNA, 23 bp, Homo sapiens
ctccacctcagacccagtactgc

SEQ ID NO: 35 - DNA, 23 bp, Homo sapiens
ccacgaagctgctgtcctcgtcc

SEQ ID NO: 36 - DNA, 23 bp, Homo sapiens
ggctgtttcctcagtcagccatg

SEQ ID NO: 37 - DNA, 23 bp, Homo sapiens
ttcctcagtcagccatgcagagg

SEQ ID NO: 38 - DNA, 23 bp, Homo sapiens
agccatgcagagggaaacccaac

SEQ ID NO: 39 - DNA, 23 bp, Homo sapiens
gtttcctcagtcagccatgcaga

SEQ ID NO: 40 - DNA, 23 bp, Homo sapiens
catgcagagggaaacccaacttt

SEQ ID NO: 41 - DNA, 23 bp, Homo sapiens
tccccgaaagaaaaagaaattta

SEQ ID NO: 42 - DNA, 23 bp, Homo sapiens
gcgagtgtgttgcaaacatgaaa

SEQ ID NO: 43 - DNA, 23 bp, Homo sapiens
aggagtcttgcctcagaatgaac

SEQ ID NO: 44 - DNA, 23 bp, Homo sapiens
accatattctaccttggtgaata

SEQ ID NO: 45 - DNA, 23 bp, Homo sapiens
gtgaataacagcgagtgtgttgc

SEQ ID NO: 46 - DNA, 23 bp, Homo sapiens
ttggtgaataacagcgagtgtgt

SEQ ID NO: 47 - DNA, 23 bp, Homo sapiens
aaccatattctaccttggtgaat

SEQ ID NO: 48 - DNA, 23 bp, Homo sapiens
ggagtcttgcctcagaatgaaca

SEQ ID NO: 49 - DNA, 23 bp, Homo sapiens
cgagtgtgttgcaaacatgaaag

SEQ ID NO: 50 - DNA, 23 bp, Homo sapiens
cagaatgaacaaccatattctac

SEQ ID NO: 51 - DNA, 23 bp, Homo sapiens
attctaccttggtgaataacagc

SEQ ID NO: 52 - DNA, 23 bp, Homo sapiens
gccagtgatgatgaaggatgtgt

SEQ ID NO: 53 - DNA, 23 bp, Homo sapiens
ctctgtgtaagggaatttactca

SEQ ID NO: 54 - DNA, 23 bp, Homo sapiens
tgctgtgatcccaaagcacatgc

SEQ ID NO: 55 - DNA, 23 bp, Homo sapiens
tccgaaggacctggtctctctgc

SEQ ID NO: 56 - DNA, 23 bp, Homo sapiens
tggacacatggcatgttccatgg

SEQ ID NO: 57 - DNA, 23 bp, Homo sapiens
gcgtctcttactactatcctaga

SEQ ID NO: 58 - DNA, 23 bp, Homo sapiens
gtctcttactactatcctagata

SEQ ID NO: 59 - DNA, 23 bp, Homo sapiens
cactgtcagtgtaaaactcatgg

SEQ ID NO: 60 - DNA, 23 bp, Homo sapiens
ggagaacaggccagtgatgatga

SEQ ID NO: 61 - DNA, 23 bp, Homo sapiens
atgcagagggaaacccaactttc